(19) United States
(12) Patent Application Publication
(10) Pub. No.: US 2009/0221936 A1
(43) Pub. Date: Sep. 3, 2009

(54) ACTIVITY-CONTINGENT WEIGHT LOSS SYSTEM

(75) Inventors: James A. Levine, Oronoco, MN (US); Barry K. Gilbert, Rochester, MN (US); Kara E. Bliley, St. Paul, MN (US); Chinmay Manohar, Rochester, MN (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: MAYO FOUNDATION FOR MEDICAL EDUCATION AND RESEARCH, Rochester, MN (US)

(21) Appl. No.: 12/303,031

(22) PCT Filed: Jun. 1, 2007
(86) PCT No.: PCT/US07/12945

§ 371 Date: May 20, 2009

Related U.S. Application Data
(60) Provisional application No. 60/810,602, filed on Jun. 2, 2006.

(51) Int. Cl. A61B 5/11

(52) U.S. Cl. 600/595

(57) ABSTRACT

An activity-contingent weight loss method and system. The system includes measuring information representative of caloric expenditure during one day, and prescribing a caloric allowance for the following day as a function of the caloric expenditure during the previous day.

### REFERENCE TO RELATED APPLICATION

This application claims the benefit of PCT/US2007/012945, filed Jun. 1, 2007, which claims benefit of U.S. Provisional Application Ser. No. 60/810,602, filed on Jun. 2, 2006, entitled Activity-Contingent Weight Loss System, which is incorporated herein by reference in its entirety.

### GOVERNMENT LICENSE RIGHTS

This invention was made with support under NIH Grant DK066270 from the National Institutes of Diabetes and Diabetes and Digestive and Kidney Diseases. The United States government may have certain rights in the invention.

### FIELD OF THE INVENTION

The invention relates to weight loss methods and activity sensors and systems used in connection with weight loss systems.

### BACKGROUND OF THE INVENTION

A large portion of the U.S. population is overweight. The health and public policy issues associated with this overweight population are significant. There remains a continuing need for improved methods and related devices and systems to assist people with weight loss and maintenance.

### SUMMARY OF THE INVENTION

The invention is an improved system and method for promoting weight control of a user. One embodiment of the invention includes receiving information representative of the user's physical activity. Information representative of the user's caloric expenditure during a past time period (e.g., today) is computed as a function of the received information representative of the user's physical activity. Information representative of a caloric allowance for a future time period (e.g., tomorrow) is computed as a function of the caloric expenditure during the past time period. The user is then provided with information representative of the future caloric allowance.

### BRIEF DESCRIPTION OF THE DRAWINGS

FIG. 1 is a flow chart illustrating steps of the activity-contingent weight control method of the present invention.

FIG. 2 is a graphical illustration of a one embodiment of a system for implementing the weight control method shown in FIG. 1.

FIGS. 3A and 3B are illustrations of pictorial, icon-based caloric allowance prescriptions that can be presented to users by the system shown in FIG. 2.

FIG. 4 is a graphical illustration of another embodiment of a system for implementing the weight control method shown in FIG. 1.

FIG. 5 is a block diagram of components of one embodiment of a sensor that can be used in connection with the systems shown in FIGS. 2 and 4.

### DETAILED DESCRIPTION OF THE PREFERRED EMBODIMENTS

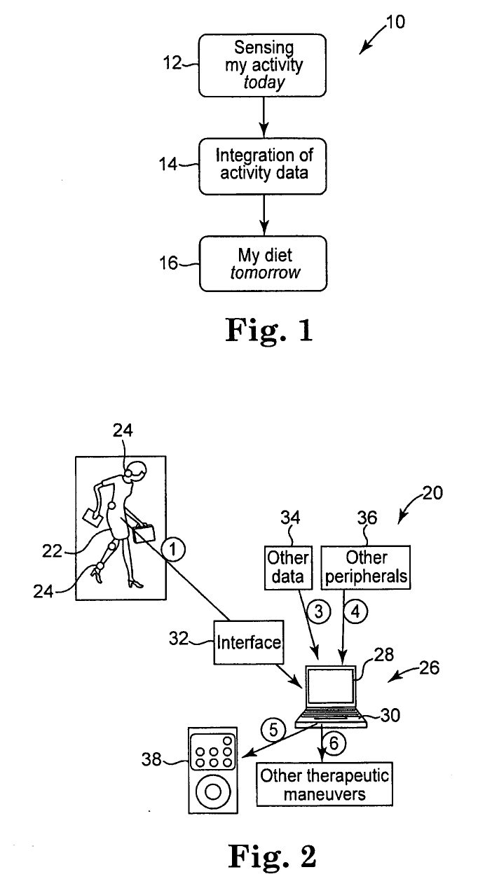

An activity-contingent weight control method 10 in accordance with the present invention can be described generally with reference to FIG. 1. Weight control method 10 is described below as a weight loss system, although the method can be used for other applications such as weight maintenance and weigh gain if desired. As shown at step 12, method 10 includes sensing information representative of the user's physical activity during a time period such as “today.” Posture and activity detectors of the type described below can be used to sense the physical activity. Non-limiting examples of the sensed activity include walking time, walking velocity and standing time. The information received from the sensor is integrated with other relevant information at step 14 to determine the caloric expenditure of the user during the time period. In one embodiment of the invention, for example, the caloric expenditure computed at step 12 includes the sum of the user's basal metabolic rate (BMR), thermic effect of food (TEF) and non-exercise activity thermogenesis (NEAT). As described in greater detail below, NEAT is computed as a function of the sensed physical activity of the user. Having determined at step 14 the caloric expenditure of the user during a past time period, the caloric allowance for a future time period such as “tomorrow” that will enable the user to loose weight is computed as shown at step 16. Information representative of the future caloric allowance can then be presented to the user. As described in greater detail below, this caloric allowance information can be presented in graphic form.

FIG. 2 is a graphical illustration of a system 20 that can be used by a user 22 to implement the activity-contingent weight loss method 10. As shown, the system 20 includes one or more physical activity sensors 24 (four are shown in the illustrated embodiments), a computer 26 having a display such as monitor 28 and a user input device such as keyboard 30, and an interface 32 for transferring information from sensors 24 to computer 26. In one embodiment of the invention the sensors 24 are posture and activity detectors (PADs) of the type described in greater detail below. Briefly, the PAD sensors 24 are relatively small, lightweight devices that can be releasably mounted to various locations on the body of the user. They include devices such as accelerometers and inclinometers for monitoring parameters of the user representative of energy-consuming physical activity. Data representative of the physical activity is stored in memory on the sensors 24. Periodically (e.g., at night or one other time per day), the data from the sensors 24 is transferred to computer 26 over data path 1 through the interface 32. In one embodiment of the invention the interface 32 is a conventional data port into which the memories in the sensors 24 can be plugged for connection to the computer 26. In other embodiments the interface 32 can be a data logger or a wireless communication system.

In addition to the activity sensed by the sensors 24, the user 22 can optionally input to computer 26 other data 34 representative of physical activity undertaken during the relevant time period. For example, the user 22 may have taken a two hour walk while the sensors 24 were not in use. The height and weight of the user 22 are also examples of other data 34. Although the entry of this other data 34 is shown being transferred over data path 3, it can alternatively be entered through keyboard 30 or another suitable interface. Data from other peripherals 36 such as a weighing scale or sensing exercise equipment can be transferred to computer 26 through data path 4.

Computer 26 can perform the activity data integration step 14 (FIG. 1) as a function of the received physical activity information. Algorithms that can be used by computer 26 for this purpose include the following.

Prediction of the calories burned today:
Basal metabolic rate (BMR): Calculated from weight, height using a formula such as: BMR=−10.6*age+8.0*weight+12.0*height−666.2, R²=0.652
Thermic effect of food (TEF): =(BMR*1.6)*0.11
NEAT. Accelerometer output can be converted to the energy cost of activity using the following algorithm.

Total daily calories expended=BMR+TEF+NEAT

NEAT Algorithms

Algorithms to convert accelerometer
output to energy expenditure:

BMR = −10.6*age + 8.0*weight + 12.0*height − 666.2 (or similar)
Sitting energy expenditure = (BMR * 1.05%)/24) * hours seated/day
Standing energy expenditure = (BMR * 1.10%)/24) * hours seated/day

| | | |
|---|---|---|
| Accelerometer whilst standing per day | 86971.48 | |
| minutes walking per day = | 430.1497 | |
| accelerometer per min of walking = | 202.1889 | |
| Accelerometer Validation/min | | |
| 0 | 7.661667 | a/min |
| 1 | 52.52321 | a/min |
| 2 | 244.9558 | a/min |
| 3 | 773.6744 | a/min |
| Slope of accelerometer and velocity | 0.003362 | |
| Intercept of accelerometer and velocity | 0.593371 | |
| solving for velocity | 1.273044 | |
| Locomotion EE above standing (kcal/min) | | |
| 1 | 1.649162 | mph |
| 2 | 2.547904 | mph |
| 3 | 3.649073 | mph |
| Slope of accelerometer and velocity | 0.999956 | |
| Intercept of accelerometer and velocity | 0.615468 | |
| solving for energy expenditure | 1.888456 | |
| walking EE per day | 812.3185 | Kcal/day |
| non-fidget NEAT = sum of walking, | 812.3185 | Kcal/day |
| sitting and standing | | |
| NEAT = non-fidget NEAT * 1.1 for | 893.5503 | Kcal/day |
| fidgeting | | |
| NEAT Gold standard value from Doubly | 875.2543 | Kcal/day |
| Labeled Water | | |

Computer 26 can also compute the caloric allowance for the following day (step 16 in FIG. 1). To lose weight, the user 22 needs to eat fewer calories than they expend. Research has demonstrated that a 30%-40% calorie reduction has substantial effects on body weight. As noted above, the caloric allowance is computed as a function of the caloric expenditure during the previous day. By way of example, for a 30% reduction, the calorie allowance for the following day (“my diet tomorrow”) can be computed as follows:

Calorie Allowance=Previous Day Calorie Expenditure*0.7

After the calorie allowance for the next day is computed the allowance is communicated to the user 22. This information can be presented to the user on the computer monitor 28. Alternatively or in addition to the presentment on monitor 28, the calorie allowance can be communicated to the user through a personal mobile communication device 38 such as a cell phone, iPOD® or BlackBerry®.

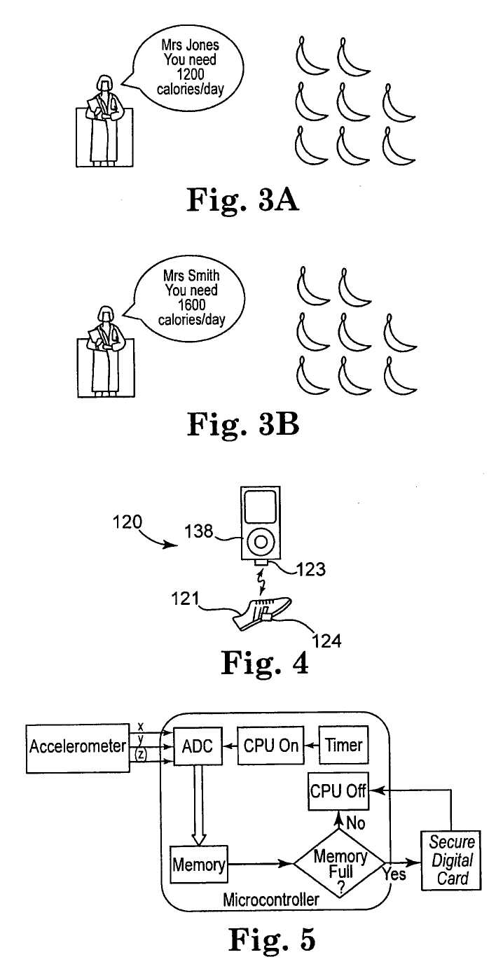

Icons or simple pictorial elements such as the apples shown in the display of communication device 38 can be used to represent the caloric allowance. FIG. 3A is an illustration of an alternative display example using six banana icons representative of 200 calorie units. FIG. 3B is an illustration of yet another display example using eight banana icons representative of 200 calorie units. The icon-based presentation of a diet prescription offers a number of advantages. It is simple to express from a software platform or by a health care provider. It is also simple for patients to understand. Calorie prescription units can be defined as recipes. Cards can be provided or electronic files generated that have recipes in multiples of the “200 calorie/banana units.” For example, a recipe for a sandwich that is “one banana unit” can be displayed. Pre-existing food items can be represented in “200 calorie/banana units.” For example, a 500 calorie hamburger cold be represented as two and one-half banana icons. The banana unit icons can also be used as a simple prescription for delivery of pre-prepared solid or liquid (shakes) food. The “tomorrow” calorie allowance can be accurately represented by the simple icon-based approach.

FIG. 4 is a graphic representation of the components of an alternative system 120 for implementing the activity-contingent weight loss method 10. As shown, system 120 includes a personal communication device 138 and an activity sensor 124. Activity sensor 124 is shown attached to a shoe 121 of a user, but can also be attached to the other clothing of the user or directly to the user's body. The activity sensor 124 is an accelerometer in one embodiment of the invention. In this embodiment of the invention the sensor 124 includes a wireless communication system for communicating information to a transceiver or receiver 123 associated with the personal communication device 138. Personal communication device 138 can also communicate with other sources of information such as computer 26 or cell phone systems. The NEAT and caloric allowance calculations can be done by a computer in the device 138, or by a remote computer (not shown) in configured for data communication with the device. Other than these differences, system 120 can function in a manner similar to that of system 20 described above.

Additional diet-related information can be conveyed to the user 22 by systems 20 and 120. This information can be provided to the user 22 through the monitor 28 of computer 26 or the display of the communication device 38. Non-limiting examples of the types of information that can be conveyed include the following.

Education—A characteristic of successful weight loss is high education level. A comprehensive text on obesity can be downloaded in pod cast format—chapter-by-chapter.

Readiness for change evaluation—Data strongly suggest that weight loss occurs when patients are “ready to make changes”. This will be evaluated on-line. With coaching for positive (continue with program) and negative outcomes (steps to take).

Learning the principles of activity contingent weight loss—Understanding activity contingent weight loss is key. Podcast, downloaded slides and video stream can be used.

Time to get going!—Showing people how the technology works show a self-testing paradigm.

Recipes—Access to downloadable recipes. Ethnicity can be built in.

Access to food items—Participants can access a catalogue of food items and their 200 calorie-Apple equivalents using the user interface (e.g., keyboard 30 or keypad of communication device 38). Restaurant items can be included.

Behavioral principles delivered weekly (pod cast)—Motivational components can be delivered on a periodic basis such as weekly. This can include music and speech content and specific behavioral elements.

Rewards delivered (e.g. download, mailed T-shirt)—Examples include a free music download per pound of weight loss. A T-shirt or reward medal for every 10 pounds of weight loss—Bronze, Silver, Gold, Platinum.

Joining the activity contingent weight loss community (personalized). Community based activity-contingent weight loss groups—both virtual and real can evolve.

Feedback loop information—If no weight loss is detected after certain time periods such as two weeks, a weight-stability algorithm can be initiated that includes a questionnaire to assess continuance. If continuance is present, the algorithms can be adjusted to decrease caloric intake (e.g. by 10%) from the current value.

A description of a PAD-type activity sensor 24 that can be used with the invention follows. Other embodiments of the invention include other activity sensors.

The key components of PAD are a 3-axis MEMS accelerometer (KXM52-1050, Kionix Inc., Ithaca, N.Y.), microcontroller (MSP430F149, Texas Instruments, Dallas, Tex.), and mini secure digital (miniSD) card (SanDisk, Sunnyvale, Calif.). These key components were selected based on their technical specifications and physical footprints. The chosen accelerometer has physical dimensions 5 mm×5 mm×1.8 mm and is one of the smallest 3-axis accelerometers available. Its power requirements are quite low at 1.5 mA (typical) at 3.3V, and when shut down, draws only about 10 uA. It still has relatively good sensitivity of 660 mV/g and noise specifications of 35 (for x and y) and 65 (for z) ug/√Hz. The acceleration range is −2 to +2 g, and meets the specifications for the intended clinical applications of PAD.

The microcontroller was chosen primarily for its very low power requirements. Its operating voltage range is 1.8 to 3.6 V. In active mode, its maximum current draw is 560 uA, and in low power mode, the typical current draw is 2 uA. The microcontroller is programmed based on interrupts and is designed to spend most of its time in very low power modes (standby) and only to become active for very short bursts when it is needed. The specific model chosen features 60 KB flash memory and 2 KB RAM with a 12-bit analog-to-digital converter (ADC).

Design requirements specified that PAD was to be completely stand-alone (not dependent on connections to a computer or other device either by wires or through wireless technology). Thus, it was necessary to identify a method of storing large amounts of data on-board the device, leading to the use of a mini secure digital card (miniSD) for data storage. The miniSD technology was chosen for its relatively small size and the variety of available storage capacities at relatively low cost (128 MB to 2 GB). PAD has been designed to be compatible with a range of cards; however, the 128 MB capacity card is sufficient for most needs.

The PAD is comprised of 113 components, including a number of supporting components that are crucial, in addition to the key components described above. One main component not mentioned previously is the clock chip (MAX6902, Maxim Integrated Products, Sunnyvale, Calif.), which is used to maintain current date and timestamp information for PAD. The date and time are sampled and stored along with each accelerometer (or other sensor) data sample. In order to maintain the clock information, extra precautions must be taken with respect to the power supply to the clock chip. In addition to the main power supply, consisting typically of 2 CR3032 coin cell batteries, a separate rechargeable lithium battery is used to power the clock chip when the main power supply is interrupted (e.g., during the replacement of the primary batteries). When the main power supply is intact, it is used to charge the secondary power supply and to power the entire system. The secondary lithium battery can power the clock chip for a few days. Another important component is the temperature sensor, which is currently used to monitor on-board temperature but could be adapted to collect ambient temperature information.

Significant time and effort were spent designing the power supply architecture. Although the system has a relatively low average current draw of 1 mA, there are periodic spikes in current requirements on the order of 80 mA, which occur when accelerometer data is written to the miniSD card. Current spikes of this magnitude necessitate the implementation of some supporting electronics even if these current requirements are only needed for few milliseconds every 16 seconds. The system voltage requirement is approximately 3 V, but in order to support both the high current spikes and meet the operating battery life requirements, the main power supply must be comprised of 2 coin cell batteries (CR3032, Panasonic Corporation, Seacaucus, N.J.), each with a voltage rating of 3.3V, arranged in series. The overall voltage of 6.6 V is down-regulated, using 2 voltage regulators, to 3.3 V. As an extra precaution, several large (680 uF) capacitors have been incorporated into the PAD to maintain a relatively constant current supply in spite of the periodic surges caused by the miniSD card.

In addition to the on-board hardware components, several peripheral supporting hardware components (a service board and remote accelerometer board) were developed for testing and support. The service board is used to download the data collection software program to the microcontroller on PAD. The microcontroller only needs to be programmed once unless the user would like to download a new software program or in case of an error. The service board features a JTAG programming interface which is compatible with the MSP430FET USB Debug interface. The service board is also used for initial functional tests performed on each newly assembled PAD, and can be used to power the PAD from either a standard power supply or AC wall plug. PAD has been designed to be compatible with other sensors in addition to the on-board 3-axis accelerometer and temperature sensor. For initial testing of expanding PAD to include a second analog sensor, a remote accelerometer board was designed, comprised of a 3-axis accelerometer identical to the one used on the PAD.

Although it has not yet been fully implemented, the peripheral accelerometer has been used in initial tests to demonstrate that PAD can be used to collect data from two independent 3-axis accelerometers simultaneously at a sampling rate of 10 Hz with full date and timestamp information.

When designing the hardware and then the outer case, a number of seemingly simple questions turned out to be quite complex. One of these design considerations had to do with the buttons and display. It is beneficial overall for the system to be simple both to minimize user confusion as well as to minimize the opportunity for errors. During the design process, it became clear that simplifying the system and protecting against user error was one of the most complex problems. After deciding that most functions could be accomplished using two buttons (even start and stop could be performed by the same button), it was necessary to design protection from unintentional button presses. The buttons were designed to be recessed into the case cover. As an extra precaution, the timing for the buttons is such that an individual needs to hold the button down momentarily. For the studies in which children wear the device, it became clear that not having access to the buttons at all is necessary. In addition to deciding how to design the user inputs to the system, it was necessary to design some simple system outputs to report device status. Is the device functioning properly or is there an error? If there is an error, what specifically has gone wrong? With a bicolor LED, there are enough different variations of display to cover most requirements, indicating proper operation and various error conditions.

The hardware components are dependent on the software to make them fully functional. The software primarily determines the PAD application. The software specifies the sampling rate, determines the data to be collected and stored, and the format of the stored data. In this section, a general description of the PAD data collection software is presented, followed by a specific description of the software program currently used in the clinical research studies conducted using PAD.

The microcontroller is programmed to sample analog data from each of the 3 axes of the accelerometer at a specified sampling rate of 10 Hz and then to perform 8-bit analog-to-digital conversion (ADC). Although the microcontroller has the capability to perform ADC up to 12 bits, it was determined that 8 bits is sufficient and that the 4 least significant bits primarily consist of noise. Also, reducing the number of bits per sample allowed a significant increase in the number of samples that can be collected before a write to the miniSD card is necessary. The sampling rate is a major factor affecting both battery life and data storage capacity. Higher sampling rates significantly reduce battery life and deplete data storage capacity at a faster rate. Laboratory testing demonstrated that PAD can collect and store 3-axis accelerometer data sampled at 10 Hz for over 14 days before a battery change is necessary. Also, using a 128 MB miniSD card provided storage capacity for over a month of this data.

Another factor affecting both battery life and data storage capacity is the data format. When designing PAD, both the ASCII and binary formats were considered. One advantage to storing data in an ASCII format is that the files can be read on a PC without any need to be converted, but the main disadvantage is that 1 byte is required for each digit. When storing data in a binary format, each 3-digit accelerometer data value is stored as 1 byte. For example, it was possible to reduce the number of writes to the miniSD card by a factor of 10, from once every 16 samples to once every 160 samples (or 16 seconds for data collected at 10 Hz). The impact on battery life is significant because as noted earlier, each write to the miniSD card spikes the current draw up to 80 mA. Therefore, minimizing the number of these current spikes is crucial in reducing the overall average current.

The current software application used in the PAD devices acquires accelerometer data (x,y,z), temperature, and voltage and stores the data to a miniSD card formatted in the FAT32 protocol. The file header, which is the information required for the data file to be recognized by a personal computer (PC), is written to the miniSD card after every 10 minutes of data collection. If some event interrupts the collection (e.g., ejection of the miniSD card or loss of battery power), the maximum amount of data lost would be merely 10 minutes worth of collection.

After data acquisition is started by pushing the start button on the PAD, the unit self-verifies that a miniSD card is in place. If a card is not detected, PAD will remain in a wait state until a card is inserted. If the miniSD card is in place, the card is read. At this time, file format information is extracted from the Reserved Region: sector size, cluster size, location of FAT, location of DATA Region, etc. All existing files and deleted files are skipped until an unused cluster entry in the FAT is found. The FAT record (sector) with the unused cluster entry is retained in an array in memory so the FAT can be filled with cluster entries as the data is collected. The FAT will be completely filled before writing the record to the card. While collecting the 16 seconds (160 sample sets collected at 10 Hz) of accelerometer data for one record (a 512 byte sector), FAT32 handling is being processed. During the collection of the 16 seconds of data, several checks occur. After collecting the first sample of the record consisting of accelerometer (x,y,z), temperature, and voltage data, information is stored in the first few bytes of the data record: device serial number, software version, record sequence number, voltage and temperature at the start of the record. After at least 2 samples of data have been collected for the record, there is a check if the data has been acquired for 10 minutes (36 records) since the beginning of the acquisition or since the last save of the File Record. If so, the FAT record is saved to the card, and then several sample sets later, the File Record is updated and saved. After collecting 8 seconds (80 samples) of data, the FAT record array is checked. If it is full, the record is written to the card. The FAT array is zeroed and the end-of-file marker is stored into the first entry of the record. After collecting the 160th sample of data, the voltage at the time of the collection of the last sample is stored at the end of the record and the data record is written to the card.

To terminate data acquisition, the stop button is depressed, turning off the accelerometer. The last full record of data and the last FAT are written to the card. Each file name is unique and contains the device serial number (3 bytes), month (1 byte), day (1 byte), hours (1 byte), minutes and seconds (2 bytes). The 32-byte “File and Directory Record” is completed by entering the number of bytes of data in the file. This record is written to the card.

When determining placement a number of different factors need to be taken into consideration. The primary consideration is that the device must be placed on the body where it will collect the most meaningful acceleration data. In addition, the attachment of the PAD at that location must be such that noise due to extraneous vibration is minimized. Placement accuracy and precision are important: the location needs to be repeatable so that data can be compared across subjects and between different time points with confidence. PAD placement must be with respect to specific anatomical landmarks that are relatively consistent between individuals. Although it would appear straightforward to place the device at the halfway point between the hip and the thigh of all individuals, the length of the arc of motion between individuals differs widely. Instead, it may be better to place the unit at a specific distance from the hip. Another consideration is that when the device is placed, it needs to remain in place, a primary issue with respect to the thigh where anatomy and gravity work against stability of the unit. For the attachment of any such device on the arm, armbands or wristbands have been employed with success, but for attachment of the device to the leg, a band does not work as well. If the band is tight enough to remain in place, it may be sufficiently uncomfortable to not be well tolerated by the subjects. Adding a band that attaches up to the waist may keep the device in place. In the laboratory environment, attachment is simple: the devices are duct-taped to the surgical scrub uniforms worn by each participant, an impractical approach outside the laboratory. For the free-living studies conducted previously by Levine et al. using devices referred to as PAMS, the accelerometers were integrated into spandex tops and shorts worn by participants under their clothing. The devices remained in place, but the concept is not likely to be accepted by the general public. Partly for this reason, the activity studies conducted using PAD have focused on the information that can be gathered by one PAD worn at the waist, which in this case was placed at the mid-lower back (belt-level). From a single PAD unit, it is possible to discern different walking speeds; the data collected in this manner also correlates well with energy expenditure data collected simultaneously. In addition to identifying methods of recording the most accurate and representative data, it is important also to explore simple methods of collecting data that is “good enough”, i.e., it is more valuable to collect some data rather than none. Even if the data could be of higher quality using a more complicated system, it is not worthwhile to develop a system that is poorly tolerated.

The most accurate measurements may be acquired if the subject is not constantly aware of the measuring device. One popular proposed method of making devices “disappear” is to integrate them into clothing. A more likely way of making the devices “disappear” is to incorporate them into an already-popular device such as a pager or cell phone, an approach that is already being investigated. Alternately, it would be relatively easy to incorporate the PAD electronics into a pager-like device, since such structures are already well accepted by the public.

Yet another embodiment of the PAD is comprised of a 3-axis+/−2 g MEMS accelerometer (Kionix Inc., Ithaca, N.Y.), a low-power microcontroller (Texas Instruments, Dallas, Tex.), and a mini secure digital (miniSD) memory card (SanDisk, Sunnyvale, Calif.). The PAD is powered by two coin cell (watch) batteries (Panasonic, Seacaucus, N.J.), which can power the unit for more than two weeks of continuous operation. The memory card used here has a storage capacity of 128 MB, allowing storage of several weeks of data (even when sampling at 10 Hz). The outer plastic case housing the unit components measures 3.5 cm×7.5 cm×1.0 cm and the complete unit, including the batteries, weighs 37 g. Each of the two batteries weighs 3.1 g, and the remaining electrical components and outer case weigh 30.8 g.

The microcontroller is programmed to control data acquisition (FIG. 5). The data acquisition software is in the C programming language using commands specific to the microcontroller. The components are in standby, or “sleep” mode—drawing minimal power unless actively acquiring data. Based on an internal timer, the microcontroller triggers each data acquisition first by turning on the CPU.

Data from the accelerometer are digitized by the microcontroller's analog to digital converter at a resolution of 8 bits and stored in onboard memory. Accelerometer data are sampled at a rate of 10 Hz in each axis of the accelerometer. The software can be modified to adjust the sampling rate from 1 Hz to 20 Hz, and can be reprogrammed to operate at even higher sampling rates. Acquired data samples are stored to the miniSD card along with an associated time-stamp. As a feature, if power to the PAD is disrupted, the data are not lost except for at most the last 10 minutes of data collected. After transferring the data to a computer equipped with a memory card reader, the data can be analyzed using a number of common software application programs. Because the data collected using PAD have associated time-stamps, data collected by multiple PAD units simultaneously can be synchronized.

The PAD data can be used to define two different types of information. In the first configuration, the PAD is used as an angle detector (inclinometer) that can measure body posture. This result is achieved by measuring the acceleration of earth's gravitational field (1 g). Acceleration measured along the two axes which are perpendicular to the axis of rotation of the body can be used to calculate the bending angle of the body using trigonometry. In the second configuration, the triaxial accelerometer is applied to detect body movement. Under this condition, acceleration is measured in all 3 axes continuously at 10 Hz.

For human subject testing, six PADs were placed on each subject: one on each side of the lateral torso, two at the base of the back, and one on each lateral thigh. The PADs were attached using standard medical tape to tight fitting undershirts and under-shorts worn by the subjects. The PAD placed on the lateral trunk was configured as an inclinometer and measured angle. The PAD placed at the lateral thigh was also configured as an inclinometer to measure angle. The x-y coordinates of the PADs placed on the torso and thigh lie in the sagittal plane, with the x-axis also in the coronal plane and the y-axis also in the axial plane. For each of the postures (lying down, sitting, and standing), the data are presented as mean and standard deviation calculated over 500 seconds. When PAD was used as an accelerometer, the x-y plane lies in the coronal plane, the y-z plane lies in the sagittal plane, and the x-z plane lies in the axial plane. The x-axis lies in the coronal and axial planes; the y-axis lies in the coronal and sagittal planes; and the z-axis lies in the sagittal and axial planes.

In summary, PAD is a small, wearable device that is robust and operates completely independent of any external infrastructure. It is a flexible platform technology that has already been used in studies on posture detection and physical activity monitoring and can be configured for use in other studies. Based on the work completed thus far, and an intense effort under way at the time of this writing to render the PAD structure more physically robust and at least water-resistant if not waterproof, PAD will soon be implemented in larger-scale studies.

Although the invention is described with reference to preferred embodiments, those skilled in the art will recognize that changes can be made in form and detail without departing from the spirit and scope of the invention.

What is claimed is:

1. An activity-based weight control system, including:
a sensor for monitoring the physical activity of a user;
a computer coupled to the sensor to:
compute information representative of a caloric expenditure of the user during a past time period as a function of the monitored physical activity; and
compute information representative of a caloric allowance for a future time period as a function of the caloric expenditure during the past time period; and
a display device coupled to the control device for providing the user with information representative of the caloric allowance for the future time period.

2. The weight control system of claim 1 wherein the computer computes the information representative of the caloric expenditure as a function of (1) the user's basal metabolic rate, (2) thermic effect of food, and (3) the user's non-exercise activity thermogenesis.

3. The weight control system of claim 1 wherein the display device provides the information representative of the caloric allowance as a graphic display of calorie units.

4. The weight control system of claim 1 wherein the display device provides the information representative of the caloric allowance as an iconic display of calorie units.

5. The weight control system of claim 1 wherein the computer further includes an input device enabling the user to input information representative of physical activity not monitored by the sensor.

6. The weight control system of claim 1 wherein the display device also provides the user with information representative of one or more of knowledge modules, behavioral modification components, rewards and recipes.

7. The weight control system of claim 1 wherein the past time period is a previous day and the future time period is a following day.

8. The weight control system of claim 1 wherein the computer computes a future caloric allowance that will cause the user to loose weight.

9. The weight control system of claim 1 wherein the sensor includes one or more posture and activity detectors that can be attached to the user,

10. The weight control system of claim 1 wherein the sensor includes an accelerometer.

11. The weight control system of claim 10 wherein the sensor includes an inclinometer to measure body posture.

12. The weight control system of claim 11 wherein the sensor further includes memory for storing data.

13. The weight control system of claim 12 wherein the sensor further includes a user operable switch for starting and stopping data acquisition and storage.

14. A method for promoting weight control of a user, including:
receiving information representative of the user's physical activity;
computing information representative of the user's caloric expenditure during a past time period as a function of the received information representative of the user's physical activity;
computing information representative of a caloric allowance for a future time period as a function of the caloric expenditure during the past time period; and
providing the user with information representative of the future caloric allowance.

15. The method of claim 14 wherein computing information representative of the user's caloric expenditure includes computing the information as a function of: (1) the user's basal metabolic rate, (2) thermic effect of food, and (3) the user's non-exercise activity thermogenesis.

16. The method of claim 14 wherein providing the user with information includes providing the user with information representative of the caloric allowance as a graphic display of calorie units.

17. The method of claim 14 wherein providing the user with information includes providing the user with information representative of the caloric allowance as an iconic display of calorie units.

18. The method of claim 14 wherein providing the user with information further includes providing the user with information representative of one or more of knowledge modules, behavioral modification components, rewards and recipes.

19. The method of claim 14 wherein the past time period is a previous day and the future time period is a following day.

20. The method of claim 14 wherein computing information representative of a caloric allowance includes computing a future caloric allowance that will cause the user to loose weight.

21. A weight control method, including:
measuring information representative of caloric expenditure during a first period; and
prescribing a future caloric allowance for a second period following the first period as a function of the caloric expenditure during the first period.

22. The weight control method of claim 21 wherein prescribing the future caloric allowance includes providing a graphic prescription representative of calorie units.

23. The weight control method of claim 22 wherein the first period and the second period are first and second sequential days.
